(19) United States
(12) Patent Application Publication
(10) Pub. No.: US 2007/0149866 A1
(43) Pub. Date: Jun. 28, 2007

(54) Systems and methods for investigation of living matter

(76) Inventors: Jerry Paul Draayer, Baton Rouge, LA (US); Hovhannes Roman Grigoryan, Newport News, VA (US); Rafik Shavarsh Sargsyan, Yerevan (AM); Sergey Armen Ter-Grigorvan, Yerevan (AM)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/656,067

(22) Filed: Jan. 22, 2007

Related U.S. Application Data
(63) Continuation-in-part of application No. 11/217,898, filed on Sep. 1, 2005.

(51) Int. Cl. A61B 5/00; G01N 21/55

(52) U.S. Cl. 600/310; 356/445

(57) ABSTRACT

Exemplary embodiments of a system for detecting biological objects are provided. In this regard, an exempliieary embodiment of such a system includes a light source, a sensor proximate to and disposed at an angle from the light source, a covering material disposed above the sensor, a photodetector proximate to the sensor and disposed at an angle relative to the light source, a non-light transmitting partition disposed between the light source and the photodetector, the partition configured to isolate the photodetector from the light source, and a non-light transmitting housing encasing the light source, sensor, and photodetector.

### CROSS-REFERENCE TO RELATED APPLICATION

This application is a continuation-in-part application that claims priority to co-pending U.S. patent application entitled, “Systems and Methods for Investigation of Living Matter,” having Ser. No. 11/217,898, filed Sep. 1, 2005, which is entirely incorporated herein by reference.

### TECHNICAL FIELD

The present disclosure is generally related to systems and methods for performing testing on living systems, and more particularly, to systems and methods for determining the presence or absence of living systems.

### BACKGROUND

In the last few decades, various devices have been developed that have applications in medicine and biology. Usually, the basic principles of work of these devices include the measurements of different physical and chemical characteristics of living systems.

The development of modern scientific principles of biological functions was essentially determined by the various instrumental methods of measuring and assessing the state of the biological functions. The instrumentation currently used in medical-biological investigations serves mostly to register and measure the physical-chemical characteristics of the living system. However, changes possibly induced in biological systems when investigating certain parapsychological phenomena (e.g., mental influences, distant healing correction, and the like) may often remain beyond limits of sensitivity of the standard apparatus.

Investigations carried out using high-voltage high-frequency methods have shown the sensitivity of Kirlian luminescence to the change of the physiological state of biological objects. Dakin, H. S. (1975), “High-voltage photography,” Published by H. S. Daskin, 3101 Washington Street, San Francisco, Calif. 94115, USA; Korotkov, K. G. (1995), “Kirlian effect,” Published by Olga, St. Petersburg, Russia (in Russia). Data obtained with these methods suggest an ability of biological systems to influence physical characteristics of gas discharge that arises around the investigated object under high-impulse voltage, such as its spatial form, intensity, and luminescence spectrum. This was clearly shown in the registration of the phantom leaf effect, where it is possible to visualize the total geometrical shape of a leaf even after a part of the leaf was mechanically removed. Choudhury, J. K., Kejiariwal, P. C., & Chattopodhyay, A., (1979). “Some novel aspects of phantom leaf effect in Kirkian photography,” Journal of the Institution of Engineers, 60 (Part EL3), 67-73. Without being bound by theory, it may thus be supposed that the effect of a high-impulse voltage includes production of ionized gas, the presence of which makes it comparatively simple to visualize such influences. Such interpretation of the mechanism of Kirlian imaging means that for detection of expected influences, in principle, another, more convenient, object can be used as a sensor.

### SUMMARY

Briefly described, embodiments of this disclosure systems and methods of detecting biological or non-biological objects. One exemplary system for detecting biological objects, among others, includes a light source, a sensor proximate to and disposed at an angle from the light source, a covering material disposed above the sensor, a photodetector proximate to the sensor and disposed at an angle relative to the light source, a non-light transmitting partition disposed between the light source and the photodetector, the partition configured to isolate the photodetector from the light source, and a non-light transmitting housing encasing the light source, sensor, and photodetector.

An exemplary system for detecting biological objects, among others, includes a light source comprising a light-emitting diode, a semiconductor laser, or an incandescent lamp, a sensor proximate to and disposed at an angle from the light source, a covering material disposed above the sensor, a photodetector proximate to the sensor and disposed at an angle relative to the light source, a non-light transmitting partition disposed between the light source and the photodetector, the partition configured to isolate the photodetector from the light source, and a non-light transmitting housing encasing the light source, sensor, and photodetector.

An exemplary system for detecting biological objects, among others, includes a light source comprising a light-emitting diode, a semiconductor laser, or an incandescent lamp, a sensor proximate to and disposed at an angle from the light source, a covering material disposed above the sensor, a video camera proximate to the sensor and disposed at an angle relative to the light source, a non-light transmitting partition disposed between the light source and the video camera, the partition configured to isolate the video camera from the light source, and a non-light transmitting housing encasing the light source, sensor, and video camera.

An exemplary method for detecting biological objects, among others, includes measuring of background light intensity of a biological measurement system comprising: a light source a sensor proximate to and disposed at an angle from the light source, a covering material disposed above the sensor, a photodetector proximate to the sensor and disposed at an angle relative to the light source, a non-light transmitting partition disposed between the light source and the photodetector, the partition configured to isolate the photodetector from the light source, and a non-light transmitting housing encasing the light source, sensor, and photodetector.

An exemplary method for detecting biological objects, among others, includes measuring of background light intensity of a biological measurement system comprising: a light source a sensor proximate to and disposed at an angle from the light source, a covering material disposed above the sensor, a photodetector proximate to the sensor and disposed at an angle relative to the light source, a non-light transmitting partition disposed between the light source and the photodetector, the partition configured to isolate the photodetector from the light source, and a non-light transmitting housing encasing the light source, sensor, and photodetector. Placing an object in non-contact proximity to the device, and viewing a video monitor to determine if the object has biological properties.

An exemplary method for detecting biological objects, among others, includes measuring of background light intensity of a biological measurement system comprising: a light source a sensor proximate to and disposed at an angle from the light source, a covering material disposed above the sensor, a video camera proximate to the sensor and disposed at an angle relative to the light source, a non-light transmitting partition disposed between the light source and the video camera, the partition configured to isolate the video camera from the light source, and a non-light transmitting housing encasing the light source, sensor, and video camera. Placing an object in non-contact proximity to the device, and viewing a video monitor to determine if the object has biological properties.

Additional objects, advantages, and novel features of this disclosure shall be set forth in part in the descriptions and examples that follow and in part will become apparent to those skilled in the art upon examination of the following specifications or can be learned by the practice of the disclosure. The objects and advantages of the disclosure can be realized and attained by means of the instruments, combinations, and methods particularly pointed out in the appended claims.

### BRIEF DESCRIPTION OF THE DRAWINGS

Many aspects of the disclosed devices and methods can be better understood with reference to the following drawings. The components in the drawings are not necessarily to scale. Moreover like reference numerals designate corresponding parts throughout the several views, unless otherwise indicated in the Detailed Description section below.

FIG. 1 is a block diagram of an example of one embodiment of the disclosed biological object measurement device.

FIG. 2 is a graph illustrating the signal-to-noise ratio of the photodetector of the exemplary device of FIG. 1 when the light source is off.

FIG. 3 is a graph illustrating the signal of the light intensity for background registration, as measured by the exemplary device of FIG. 1.

FIG. 4 is a graph illustrating the signal of the light intensity for various objects placed at a distance, as measured by the exemplary device of FIG. 1.

FIG. 5 is a block diagram of an example of another embodiment of the disclosed biological object measurement device.

FIG. 6 is a graph illustrating the signal of the light intensity for a human palm placed at a distance, as measured by the exemplary device of FIG. 5 when an aluminum partition is used.

FIG. 7 is a graph illustrating the signal of the light intensity for a human palm placed at a distance, as measured by the exemplary device of FIG. 5 when no partition is used.

FIG. 8 is a graph illustrating the signal of the light intensity for a human palm placed at a distance, as measured by the exemplary device of FIG. 1 when no amplifier is used.

FIG. 9 is a block diagram of an example of another embodiment of the disclosed biological object measurement device.

FIG. 10 is a graph illustrating the signal of the light intensity for a heated non-biological object placed at a distance, as measured by the exemplary device of FIG. 9.

FIG. 11 is a graph illustrating the signal of the light intensity for a human palm placed at a distance, as measured by the exemplary device of FIG. 9.

FIG. 12 is a block diagram of an example of another embodiment of the disclosed biological object measurement device.

FIG. 13 is a graph illustrating the signal of the light intensity for a warm non-biological object placed at a distance, as measured by the exemplary device of FIG. 12.

FIG. 14 is a graph illustrating the signal of the light intensity for a human palm placed at a distance, as measured by the exemplary device of FIG. 12.

FIG. 15 is a block diagram of an example of another embodiment of the disclosed biological object measurement device.

FIG. 16 is a graph illustrating the signal of the light intensity for a human palm (1) and a heated non-biological object (2) placed at a distance, as measured by the exemplary device of FIG. 15, where the registration is in plane, parallel to the incident light direction.

FIG. 17 is a graph illustrating the signal of the light intensity for a human palm (1) and a heated non-biological object (2) placed at a distance, as measured by the exemplary device of FIG. 15, where the registration is in mutually perpendicular planes.

FIGS. 18A and 18B are graphs illustrating the signal of the light intensity for temporary “biologization” of a non-biological object, as measured by the exemplary devices of FIGS. 1 and 15 respectively, before (1) and after (2) being placed in contact with a biological object,

FIG. 19 is a block diagram of an example of another embodiment of the disclosed biological object measurement device.

FIG. 20 is a graph illustrating the background signal with a transistor laser as the source of light.

FIGS. 21a and 21b are graphs illustrating the signal of the light intensity as influenced by living objects.

FIG. 22 is a graph illustrating the signal of the light intensity as influenced by a heated object.

FIGS. 23a and 23b are graphs illustrating the signal of the light intensity for temporary “biologization” of a non-biological object before (23a) and after (23b) being placed in contact with a biological object.

FIG. 24 is a graph illustrating the signal of the light intensity as influenced by mechanical stimulation of a living object.

FIG. 25 is a graph illustrating the signal of the light intensity as influenced by thermal changes to a biological object (leaf).

FIG. 26 is a block diagram of an example of another embodiment of the disclosed biological object measurement device.

FIG. 27 shows six representations (Frames A-F) of the output of the embodiment of the device in FIG. 26.

FIG. 28A shows a graph of the output of system 200 wherein the arrow indicates when a human enters the room with the device.

FIG. 28B shows a graph of the output of system 200 beginning when the human departs the room.

### DETAILED DESCRIPTION

Embodiments of the present disclosure include biological object measurement devices, and methods and systems that utilize alternative methods for evaluation of biological systems without direct and/or physical contact. In the detecting state, embodiments of the present disclosure register an anomalous influence of living systems in a manner not explained by traditional methods of sensor activation (heat transfer, electromagnetic radiation, mechanical interaction, etc.). Embodiments of the present disclosure uniquely demonstrate the ability of biological systems to remotely qualitatively and quantitatively affect the sensor.

Biological Obiect Measurement Device Hardware

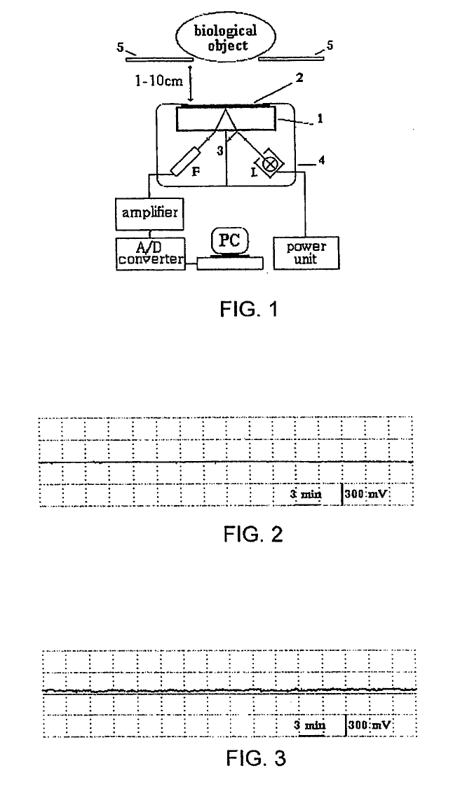

The operation of embodiments of the present disclosure is based, at least in part, on the level of light reflection by a sensor made of a glass plate covered with an opaque material. FIG. 1 shows an illustration of one embodiment of the disclosed biological object measurement device, which can also be described as a biological system detector. In general, embodiments of the present disclosure are based on the level of light reflection by a sensor made of a glass plate covered with an opaque material. Radiation energy (e.g., microwaves, infrared (IR) light, ultraviolet (UV) light, visible light, and x-rays) from a light source L is proximate to and directed, preferably at an angle, at a sensor 1. The radiation energy is refracted by the sensor 1 and reflected from an upper inside surface of the sensor 1, and then falls upon a photodetector F, or is partially reflected from a lower surface of the sensor 1. The photodetector F measures the total intensity of incoming light and is disposed proximate to the sensor 1. A partition 3 isolates the photodetector F from the source of light L and from the light reflected by a lower surface of the sensor 1. The light source L, the sensor 1, and the photodetector F are completely isolated from external light by a non-light transmitting housing 4 (e.g., a metallic case). A portion of the light goes out of the bounds of the upper surface of the sensor 1, and is then either absorbed by a covering material 2 and/or is reflected from the covering material 2, after which it also falls upon, and is detected by, the photodetector F.

Additionally, an optional temperature-controlled power unit can be used for ensuring the stability of the radiation level of the light source L. An optional differential amplifier (e.g., with a band-pass up to 20 Hz) can be used to increase the noise-immunity of the device. The amplifier can be controlled by a computer processing system, such as, for example, a PC or other electronic display/storage system with an optional A/D converter.

Materials and Methods

In one embodiment, the light source L can include, but is not limited to, light-emitting diodes operating in different spectral domains, semiconductor lasers, microwave emitters, ordinary incandescent lamps and combinations thereof. In an embodiment in which the light source L is an incandescent lamp, the radiation spectrum of the light source L is about 400-3000 nm with a peak intensity at about 1000 nm. The sensor 1 can be, for example, a glass plate, a polyethylene material, another type of plastic material, a cardboard paper, an opaque material, or other material that can refract and a reflect light. Specifically, the sensor 1 can be a glass plate of about 2-4 cm in width. The sensor 1 can also be a glass plate of about 2 cm in width. A paper sensor can be about 0.01-0.1 cm in width. A cardboard sensor can be about 50-100 microns (μm) in width. The width of the sensor 1 can be chosen to improve the effect measured by the disclosed device.

The covering material 2 can be an opaque material, such as, but not limited to, a dense black paper, a cardboard paper, a thin non-transparent black plastic, and the like. For the opaque covering, thin opaque plastic materials and black cardboard with a thickness of about 50 to 100 μm can be used. The photodetector F can include, but is not limited to, photomultiplier tubes, vacuum tubes, and semiconductor photocells.

The impact angle of the light rays from the light source L onto the sensor 1 can be varied. For example, the impact angle can range from about 40° to 60°. The diameter of the most illuminated part of the upper surface of the sensor 1 can be varied from about 1 to 4 cm.

The standard method of subtraction of the steady component from the photodetector signal was used to remove the reflected light intensity. After the amplification (up to about 500 times) of this difference, the signal was fed to an analog-to-digital (A/D) converter, and the digitized data were then sent to a PC or other electronic processor system for processing and/or display/storage. The value of the steady component was defined by the complete visualization of all changes of signal amplitude on the monitor. The value of the steady component was adjusted such that all changes of signal amplitude could be completely visualized on the PC's monitor.

The level of intrinsic noise of the disclosed biological object measurement device was tested, and found not to exceed about 0.008 mV. A 16-bit A/D converter with a conversion time of 0.6 ms and a quantization step of 0.25 mV was used. The program package was developed for that purpose, and the processing speed of the PC made it possible to measure the current value of the intensity of the light entering the photodetector with sampling step of 25 ms. The series of reflected light intensity measurements was smoothed, using the moving average method with the sample step of 25 ms and averaging time of 2.5 s (e.g., with sliding window length 100 samples). The averaged values of the measured signal were displayed every 25 ms. The stability level of the background signal can be estimated using the graph shown in FIG. 2, which shows the recording of the photodetector indication at the inactive radiation source.

After recording the control level of the background intensity of the light reflected by the sensor 1 of the device when no biological object is present, the investigated biological object was disposed on the rack that was previously placed at a distance from about 1 to 10 cm from the device sensor 1, and the character of change of the registered signal was assessed. Apples, grapefruits, and laboratory animals (e.g., rats) were used as biological objects. Before the tests, rats were subject to NEMBUTAL® anesthesia, using 50 mg/kg dosage. Tests were also carried out with participation of human subjects.

In FIG. 3, an example is shown of a prolonged background registration of the reflected light intensity. Using the mean value of the background level intensity (I_{back}), it was estimated what level the mean value of the registered signal (I) has to affain in order to allow for a conclusion that a change of the reflected light intensity really occurred. For that reason, the Studentized t test was used,
t=|/_{back}−/|/(S²_{back}/N_{back}+S²/N)^{l/2},
where S_{back}, S, N_{back}, and N, respectively, denote root-mean-square deviations and numbers of measurements carried out for sections of recording to be compared. With 10-s measurement intervals, the number of data points collected was N_{back}=N=400. After amplification, the maximum amplitude excursion for registered signal background oscillations did not exceed 20-60 mV. Even if, for root-mean-square deviations, one uses the value of the maximum excursion of oscillations of the background part of the curve (equal to 60 mV) than at significance level α<0.001 (when t=3.3), the intensity |/_{back}−/|≈6 mV was obtained. In this test, the deviation of the signal from the mean level of background oscillations usually exceeded the maximum excursion of background oscillations about 5 to 10 times, which provided a high level of reliability of observed effects. Taking this into account, single tests are illustrated, without statistical analysis of data for one-type tests.
Results and Discussion

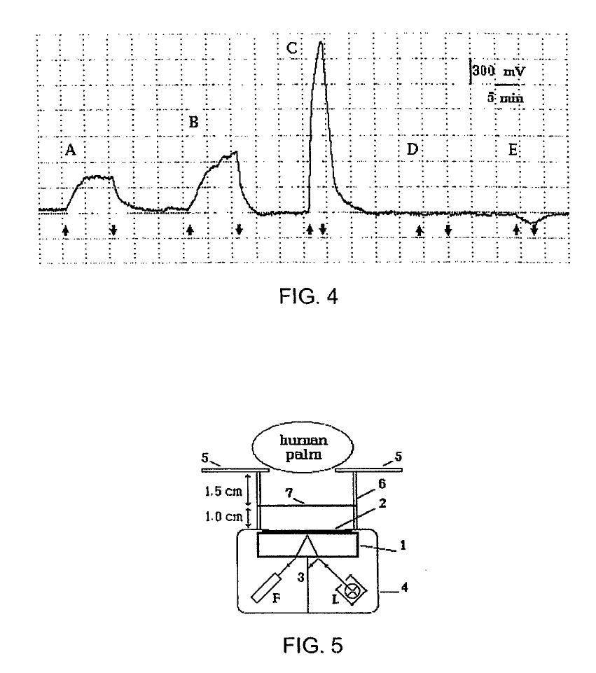

FIG. 4 represents the graphical results of the distant influence of different objects on the indications provided by the disclosed biological measurement device. It was found that after about 10 to 100 s after the biological object is placed on a rack 5 (see FIG. 1), affixed at a distance ranging from about 1 to 10 cm from a top surface of the sensor 1 of the disclosed device, a reliable change of intensity of the reflected light of the light source L is observed by the sensor. With respect to the objects studied in FIG. 4 using the disclosed device, the distance of all objects from the sensor 1 is about 1 cm. It is observed that after about 10-100 seconds, certain biological objects initiate a significant change in the registered signal from the photodetector, relative to the background noise level.

In all figures, the deviation of the curve upward corresponds to increasing intensity of the light reflected by the sensor. Arrows in FIG. 4 indicate the approaching and removing of the investigated object in relation to the sensor 1. In FIG. 4, peaks A, B, and C are examples are presented of such influences caused by an apple, a grapefruit, and a human palm, respectively.

The magnitude of the effect differs for various biological objects. In the case of the human palm, the increase of the reflected light intensity can amount to about 1% to 2% of the absolute value of control level of the registered signal. After the biological object is removed from the rack 5, the amplitude of the registered signal returns to the control level. If the distance from the biological object to sensor is increased, the time during which the effect occurs increases, and the change of reflected light intensity itself is diminished.

In living or biological systems, the stability of biological functions, energetic equilibrium, and continuous interaction with the environment is maintained by a variety of biochemical reactions. In all of these processes, electromagnetic interactions may be important. Electromagnetic fields generated by biological systems and detectable in their environment are too weak and of too low frequencies, so they cannot affect the sensor in such a way that the intensity of the reflected light would change. We have also tested this directly, generating by physical means electromagnetic fields of much higher intensity than are typical intensities of electromagnetic fields of living systems. Despite the high intensity electromagnetic field, there is no detectable influence on the readings of the disclosed device.

Further, the temperature of investigated biological objects was equal or higher than the environmental temperature to demonstrate that temperature and/or heat exchange does not influence the readings of the disclosed biological object measurement device. Nonliving objects (e.g., metal, glass, plastic, and the like), having environmental or room temperature, do not influence the value of the registered signal. For example, an aluminum plate at environmental temperature was tested with the disclosed device. As can be seen in area “D” of FIG. 4, there was no change in measured intensity.

The control tests performed using the disclosed device show that the identical but heated objects cause the decrease of the reflected light intensity. For example, as can be seen from the inverse peak “E” of FIG. 4, the reflected light intensity decreased when the same plate used for area “D” was heated to about 40° C. Thus, the possibility of an influence of biological systems on the sensor by heat radiation is also excluded.

Due to processes of gaseous exchange and evaporations, a peculiar chemical “micro-atmosphere” is formed around biological objects. To demonstrate that chemical interactions do not influence the device, control methods were carried out in which an immediate contact of the objects with the surface of the sensor 1 was avoided (see FIG. 5). In this case, the lower part of the sensor 1 was physically isolated from the environment. Included in the embodiment of the device of FIG. 5 are metallic tubes 6 closely fitting to the main body of the device. In the embodiment of the device of FIG. 5, the diameter of the metallic tube was about 4 cm and the wall thickness was about 4 mm. Also included in FIG. 5 is a plate 7 hermetically built in the tube 6 and isolating the sensor 1 from the environment. Thus, through mechanical isolation of the system, the possibilities of chemical and mechanical (vibration) interactions have been experimentally eliminated as potential causes of the observed response of the detector to biological systems.

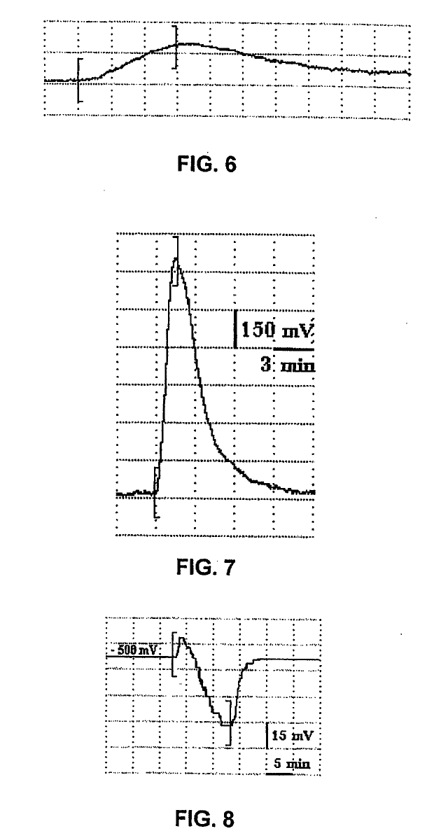

Shown in FIGS. 6-8 are the results of the influence of the palm on the reflected light intensity. In each of the tests the results of which are depicted in FIGS. 7-8, the distance from the palm to the sensor surface was about 2.5 cm. The brackets represent the time interval of the influence.

In FIG. 6, the plate 7 used was an aluminum foil with a thickness of about 0.05 mm, coated from both sides by a thin layer of a polyethylene. As can be observed from FIG. 6, even after a hermetic isolation of the outer side of the sensor 1 from the ambient atmospheric environment, a well-pronounced effect can be observed, when the human palm is placed at a distance of about 2.5 cm from the surface of the sensor. The magnitude of the influence decreased somewhat when thicker and denser materials were used for encapsulation of the sensor (see FIG. 6). FIG. 6 shows the results for testing of the human palm when the plate 7 is an aluminum foil partition with a thickness of about 0.05 mm, coated on both sides with a thin layer of a polyethylene. Similar results were obtained when using the embodiment of the device of FIG. 5, except with a tin plate with a thickness of about 0.1 mm was used for the plate 7 instead of the aluminum partition.

In FIG. 7, for comparison, analogous data are presented at the absence of the isolating the partition 7. FIG. 7 depicts the result of a control registration of the palm influence on the reflected light intensity at the absence of the plate 7.

In some embodiments of the devices and methods, depending on the selection of the covering materials, the effects from approaching biological objects to the biological measurement device 10 may change (decreasing about 7% to 8%) relative to the control level. FIG. 8 demonstrates the two-component device signal caused by a human's palm approaching. The registration was carried out in a direction from the photodetector F, without using the amplifier. It was presented as the absolute value of the initial output voltage of the photodetector. The brackets “[ ]” indicate the time interval during which the palm was disposed at a distance 1 cm from the device sensor 1.

Additional Tests

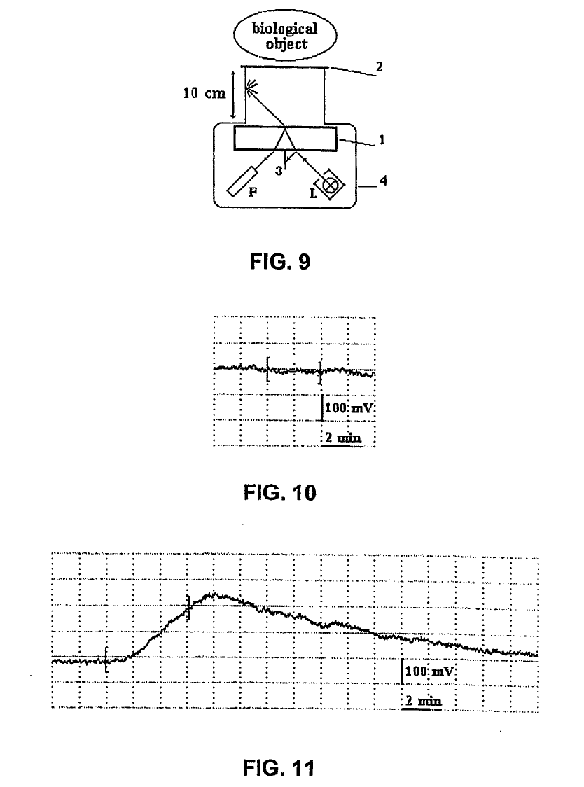

Effects observed may be conditioned by the change of physical parameters of the glass plate and covering material. In order to account for its role in the formation of observed phenomena, the following test was carried out: the covering material was moved away from the glass plate at such a distance, that light reflected from it did not influence photodetector indications. The test was performed in the absolute darkness. One embodiment of the biological object measurement device used to perform the test is depicted in FIG. 9.

FIG. 10 shows the results of the influence of a heated lifeless object, with the object being heated to a temperature of about 40° C. As can be seen in FIG. 10, the approaching of the warm (e.g., about 37-40° C.) object did not change the level of intensity of measured light.

FIG. 11 shows the results of the influence of a human's palm, at a temperature of about 34-35° C. As can be seen, the approaching of the palm after 1-1.5 min caused the gradual formation of the effect. About 25-30 min after removing the palm, the reflected light intensity was restored to the initial level.

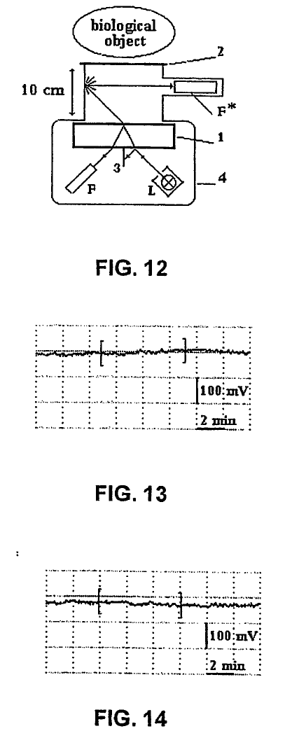

For the estimation of possible change in intensity of passing through the sensor 1, three light tests were performed using the embodiment of the device shown in FIG. 12. As shown in the embodiment depicted in FIG. 12, the sensor 1 can be configured to allow some light to pass through and be reflected from a second surface in the housing 4 that is perpendicular to an upper surface of the sensor 1. An additional photodetector F* is disposed in the same plane as the light reflected from the second surface. With the help of the photodetector F*, the intensity of the luminance was estimated in the region of the light spot on the inside surface of the non-light transmitting housing or case 4. It was found that no effects are observed in both of the cases of the warm (e.g., about 40° C.) lifeless object (FIG. 13) and biological object (e.g., a human's palm, the results of which are shown in FIG. 14). Thus, the intensity of light, reflected from the upper surface of sensor 1, and the intensity of light passing through the sensor 1 is not changed. The observed phenomena by the biological measurement device are therefore not connected with the change of the optical density of the glass plate, changes of the reflectance and absorption coefficient of its surface, or any micro-deformation processes in it.

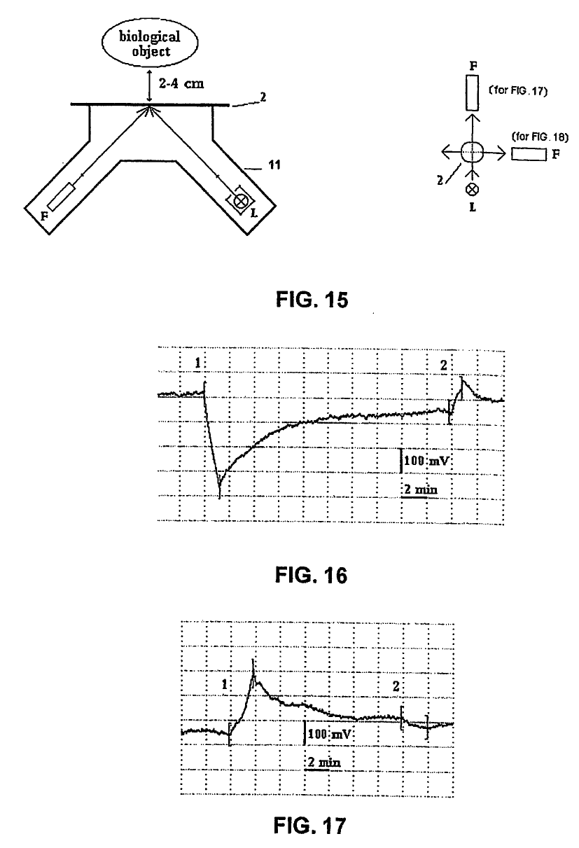

Measurements were also taken with an embodiment of the disclosed device that registers the intensity of light reflected from the covering material 2. As in FIG. 15, an embodiment of the device was tested without the sensor 1 in a chamber 11 of the device that is completely closed off from external light. The intensity of light reflected from the covering material in different directions was measured. In the embodiment of the biological object measurement device shown in FIG. 15, the light source L and photodetector F were disposed both in one plane and in mutually perpendicular planes. As shown in FIG. 16, when the light source L and photodetector F are disposed in one plane, when the human palm approaches the covering material, at a distance of about 2-4 cm a decrease is observed of the intensity of light reflected from it. Also in FIG. 16, for lifeless objects there is an increase of the intensity. When the light source L and photodetector F are disposed in perpendicular planes, the reverse pattern of light intensity is measured, as depicted in FIG. 17.

Analogous effects are observed also for other biological objects, while they are absent for lifeless objects at the environmental temperature. In the palm-approaching tests, the change of light intensity reflected from the covering surface may reach a few percentage points relative to the absolute value of the control level from photodetector signals. Effects from fruits are expressed weaker, and do not exceed about 1%.

Even in simplified tests, biological objects show a reliable change in the character of reflected light from the covering surface. The influence of a warm lifeless object has the inverse direction, which includes a change of the angular distribution of scattered light intensity.

In the initial scheme of the above-mentioned tests for using the biological measurement device, effects on light intensity are present as a result of summation of the change in the reflected light intensity from the upper surface of sensor 1 and from the covering material 2. The difference in amplitude-time characteristics of the effects formation could lead to the two-component shape of the registered signal.

The embodiment of FIG. 15 can be used to carry out testing of various types of materials. The expressiveness and the stability of effects depend on the type of the covering material. The change of the angular distribution of the reflected light intensity is not symmetric and depends on the covering material orientation.

Results

The biological object measurement device clearly demonstrates the ability of living systems to exert distant influence on the environmental objects, as evidenced by the fact that the device indications change if one places the biological object near the sensor.

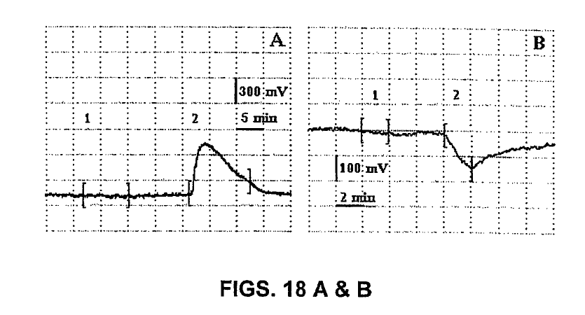

The nature of investigated remote interaction of biological objects is demonstrated by the following phenomenon. It was found that after being in close proximity to biological objects for a few minutes, some non-living materials (e.g., paper, wood, glass), which at first did not cause any effect, temporarily acquired the possibility to change the intensity of the reflected light from the sensor. FIGS. 18A and 18B illustrate the results of the effect of temporary “biologization” of non-living objects. FIGS. 18A and 18B depict the results obtained from embodiments of the disclosed device (as shown in FIGS. 1 and 15 respectively) without using the sensor 1. In both FIGS. 18A and 18B, the numeral “1” represents the control area for registration of influence of a thick piece of paper (the non-living material studied). The area designated by the numeral “2” demonstrates the measurement of the exact same piece of paper after it has been held for 2 min between a human's palms. In both FIGS. 18A and 18B, the distance from the sensor was about 2 cm.

It is clear from FIGS. 18A and 18B that the directness of change in reflected light's intensity is the same as in the case of biological objects. After close interaction between the biological object and a non-living object, certain changes occur in the condition of the non-living object. The non-living object became “biologized” in that manner that can be registered or measured by the disclosed device. The time during which the change entirely vanishes may amount to about 15-30 min. An effect is also observed even when there is no contact between the biological object and the non-biological object. In one example, the palms of the human hands did not touch the paper, and were located at a distance about 4-5 mm from it before the paper was inserted into the sensor and the results were observed. The change was temporary and lasted from about 10-30 minutes.

In testing of narcotized rats, it was shown that after an injection of a lethal dose of Nembutal® sedative anesthetic compound, from Abbott Laboratories Corp. of Ill., USA, a decrease of the registered signal level was observed. The indication of the disclosed device reflects the level of the biological activity of the living system. By the amplitude of the deviation from the control level, the investigated biological object's functional state can be judged without even contacting the biological object. Thus, the disclosed device may be used as an instrument for a new non-contact method of estimation of the functional state of biological objects.

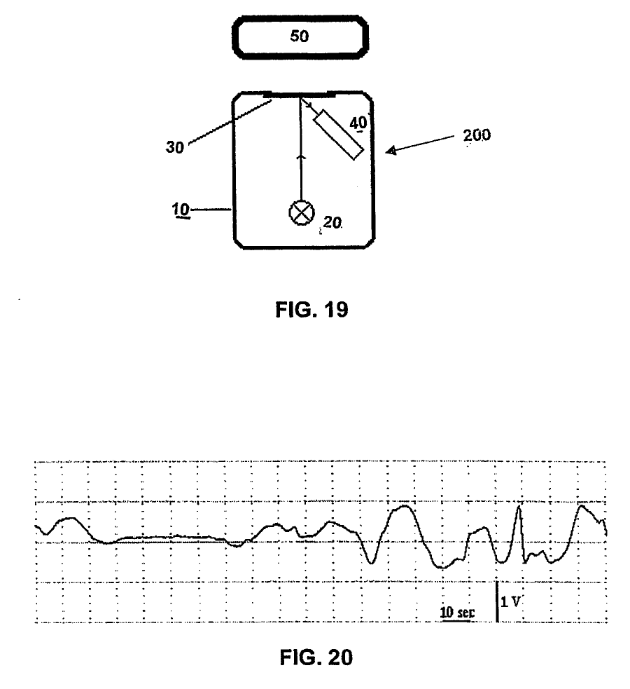

FIG. 19 illustrates an embodiment of the disclosed system 200. The results of the tests conducted with the device of FIG. 19 are demonstrated in FIGS. 20-25. In FIG. 19, the housing 10 encases a light source 20, a sensor 20, and a photodetector 50. The object 5 being measured is located outside of the housing 10. In one embodiment shown in FIG. 19, the housing 10 is a metallic frame, the light source 20 is a transistor laser (e.g., wavelength of about 0.63 μm), the sensor 30 is a mat plate covered with a light insulating material (e.g., black paper), and the photodetector 50 is a photodiode (e.g., φ256).

In tests conducted with the system 200, electronic transformation and visualization of the signal on the monitor are performed similar with the case of an ordinary lamp. However, the registered signal here is not smoothened but only averaged during 10 ms. As seen in FIG. 20, the background signal of photodiode in this case contains irregular oscillations with frequency less that 0.1 Hz. FIG. 20 depicts the recording of background signal with transistor laser as the source of light.

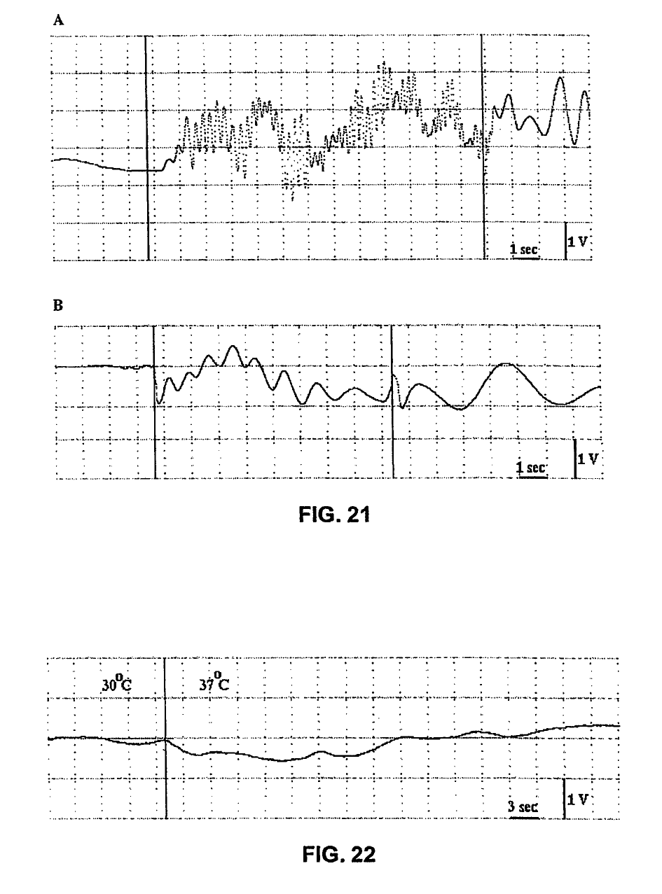

It is shown that the approach of the living objects near the detector causes the formation of characteristic and relatively high frequency signals (up to 10 Hz). The amplitude of these oscillations can reach about 7-10% from the absolute value of the background signal of photodiode. FIG. 22 illustrates the influence from a heated object, where the vertical line denotes the moments of approach and removal of the biological object to the housing 10.

After the living objects are removed from the proximity of the detector the frequency of oscillations lower and after about 5-10 min the oscillations return to their initial low frequency irregular behavior.

To exclude the thermal factors in the formation of these high-frequency signals, the following control experiments were done. A glass made of thin metallic frame and half filled with water at room temperature is placed near the detector. During the registration the small portions of hot water were added in the glass. It may be seen from FIG. 22 that the heating of the glass does not cause to any substantial change in the character of the signal. In FIG. 22, the vertical line denotes the moment of the object's temperature increase.

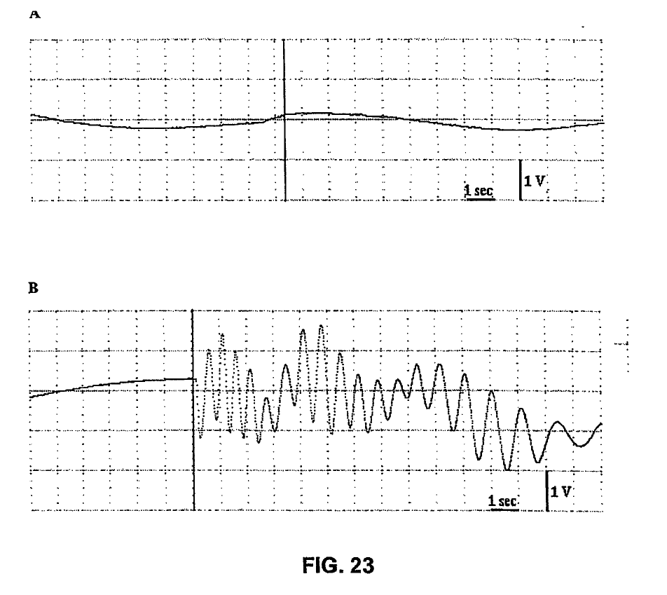

It is also shown that using this type of registration method produces an effect on the lifeless object described earlier as “biologization”. It is shown in FIG. 23A that the approach of dense piece of paper does not change the registered signal. After it was placed between the palms for one minute, the approach of the paper to the detector creates the formation of oscillations (FIG. 23B). The vertical line in FIGS. 23A and 23B denotes the moment of approach.

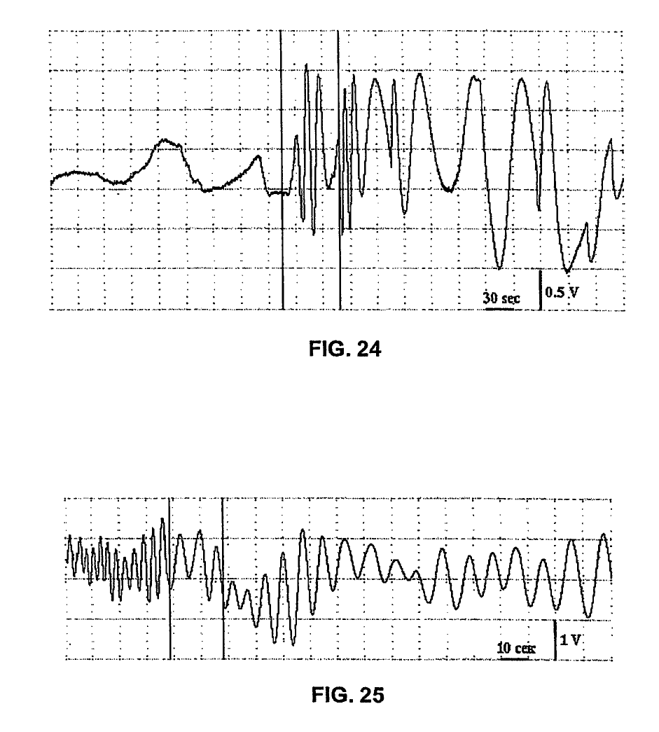

FIG. 24 illustrates a test of the embodiment of FIG. 19 on a rat narcotized with urethane medication. During the rat's anesthesia the high frequency oscillations that are characteristic to living systems is not observed. However, the high frequency and large amplitude oscillations form after mechanical stimulation of the rat. These oscillations occur for a long time even after the stimulation is removed. In FIG. 24, the vertical lines denote the beginning and end of the mechanical stimulation.

As shown in FIG. 25, the initial oscillations (with frequency about 0.3 Hz) of the signal registered in the region of one leaf of the plant substantially decreases after the thermal influence on the different leaf of the same plant. The vertical lines in FIG. 25 denote the moments of thermal influence.

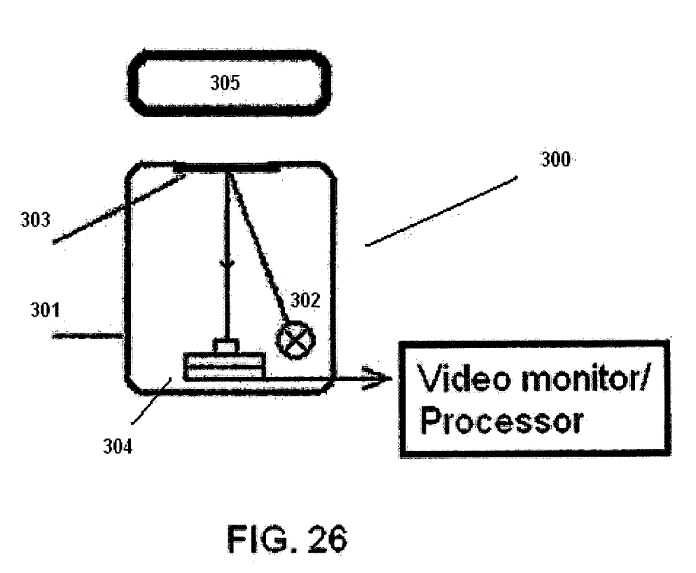

FIG. 26 illustrates an embodiment of the disclosed system 300. In which a metallic cover 301 encases a diode laser 302, a sensor 303 including a maft glass plate covered with black paper, and a video camera 304. A biological object 305 being measured is located outside of the metallic cover 301. The embodiment uses video camera 304 to detect the light emitted from diode laser 302 and sends the results to a video monitor or processor 305.

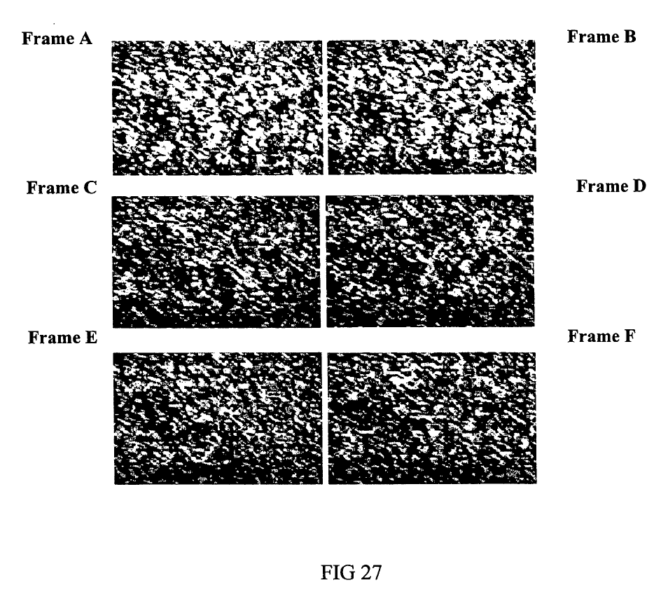

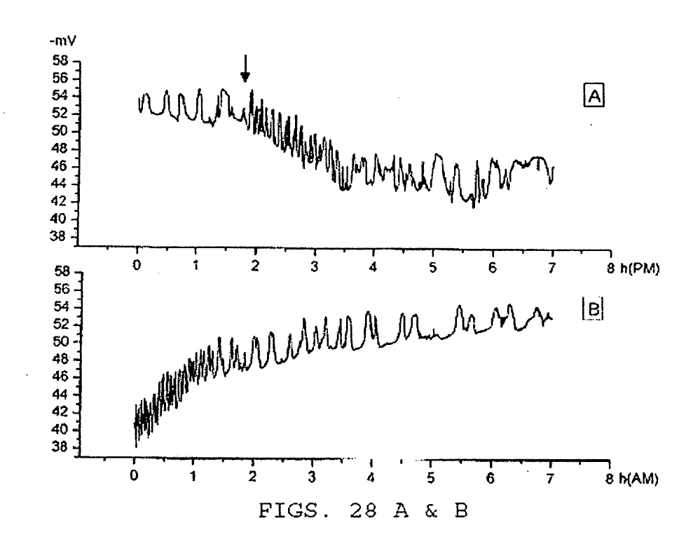

The results of the tests conducted with the device are shown in FIG. 27. Frames A and B are frames that display the still shots of video output of the device without a biological object present. Frames C, D, E, and F illustrate the dynamic change in the video pattern as a human palm is moved into the vicinity of the device.

FIG. 28A shows a graph of the output of system 200 wherein the arrow indicates when a human enters the room with the device. One may note that the voltage values over time decrease once the room is occupied. FIG. 28B shows a graph of the same system 200 beginning when the human departs the room. The voltage levels begin to increase once the room is vacant.

The results of the tests using embodiments of the device that included a diode laser resulted in readings that indicated the presence of biological matter that was in a distance in excess of three meters.

The use of the laser with a video camera and monitor in place of a photo detector, provided a visual effect evident to the naked eye when biological matter was present in the same room as the device.

A possible use of many embodiments of this device would be as a security device, which could output either a digital or analog signal to a processor or logic device to detect the presence of biological matter in an area.

Further, a processor could use the video output in automated systems which use video signals to direct the system, such as robotic manipulators. Many logic routines that use visual images of objects to recognize and identify them. Such a device could greatly simplify the logic routine by allowing a processor to easily determine whether an object has biological properties without having to analyze its shape.

Biological objects are complex systems. The tests in this study showed that the directions of the change of the reflected light intensity caused by biological objects and heated nonliving objects are always opposite. Nevertheless, the influences of warm objects exist, and they are analogous to “biological influences.”

It should be emphasized that the above-described embodiments of the biological object measurement devices and methods are merely possible examples of implementations of the devices and methods, and are merely set forth for a clear understanding of the principles set forth herein. Many variations and modifications may be made to the devices and methods without departing substantially from the spirit and principles of the disclosure. All such modifications and variations are intended to be included herein within the scope of this disclosure and protected by the following claims.

At least the following is claimed:

1. A biological measurement system comprising:
a light source;
a sensor proximate to and disposed at an angle from the light source;
a covering material disposed above the sensor;
a photodetector proximate to the sensor and disposed at an angle relative to the light source;
a non-light transmitting partition disposed between the light source and the photodetector, the partition configured to isolate the photodetector from the light source; and
a non-light transmitting housing encasing the light source, sensor, and photodetector.

2. The system of claim 1, further comprising:
a power unit electrically coupled to the light source;
an amplifier for the photodetector signal electrically coupled to the photodetector;
an analog-to-digital converter electrically coupled to the amplifier; and
a display/processor system communicatively coupled to at least one of the photodetector, the amplifier, or the converter.

3. A biological measurement system comprising:
a light source selected from: a light-emitting diode; a semiconductor laser; and an incandescent lamp;
a sensor proximate to and disposed at an angle from the light source;
a covering material disposed above the sensor;
a photodetector proximate to the sensor and disposed at an angle relative to the light source;
a non-light transmitting partition disposed between the light source and the photodetector, the partition configured to isolate the photodetector from the light source; and
a non-light transmitting housing encasing the light source, sensor, and photodetector.

4. The system of claim 1, wherein the sensor is chosen from at least one of the following: a glass plate, polyethylene film, a plastic material, a cardboard paper, and a material that refracts and reflects light.

5. The system of claim 1, wherein the sensor is chosen from at least one of the following: a glass plate, polyethylene film, a plastic material, a cardboard paper, and a material that refracts and reflects light.

6. The system of claim 1, wherein the sensor is chosen from at least one of the following: a glass plate of about 2-4 cm in width, a paper sensor of about 0.01-0.1 cm in width, and a cardboard sensor of about 50-100 microns in width.

7. The system of claim 1, wherein the covering material is chosen from at least one of the following: a dense black-colored paper, a cardboard paper, and a thin non-transparent black-colored plastic.

8. The system of claim 1, wherein the photodetector is chosen from at least one of the following: photomultiplier tubes, vacuum tubes, and semiconductor photocells.

9. A biological measurement system comprising:
a light source;
a sensor proximate to and disposed at an angle from the light source;
a covering material disposed above the sensor;
a video camera proximate to the sensor and disposed at an angle relative to the light source;
a non-light transmitting partition disposed between the light source and the video camera, the partition configured to isolate the video camera from the light source; and
a non-light transmitting housing encasing the light source, sensor, and video camera.

10. The system of claim 1, further comprising:
a tube that extend from the housing perpendicular to the sensor; and
a plate built inside the tube that is parallel to the sensor, wherein the plate isolates the sensor from surrounding environment.

11. The system of claim 1, further comprising a surface of the housing disposed perpendicular to the sensor on an opposite side of the sensor from the light source and photodetector, the surface arranged to reflect light that passes through the sensor.

12. The system of claim 11, further comprising a second photodetector disposed in an identical plane as the light reflected from the surface of the housing that is perpendicular to the sensor.

13. A method of measuring the activity of a biological object comprising the steps of:
measuring a background light intensity of a biological measurement system comprising:
a light source;
a sensor proximate to and disposed at an angle from the light source;
a covering material disposed above the sensor;
a photodetector proximate to the sensor and disposed at an angle relative to the light source;
a non-light transmitting partition disposed between the light source and the photodetector, the partition configured to isolate the photodetector from the light source; and
a non-light transmitting housing encasing the light source, sensor, and photodetector;
placing an object in non-contact proximity to the device; and
measuring a light intensity from the sensor of the device.

14. The method of claim 13, further comprising the step of:
determining that the object is a biological object by measuring a change in the light intensity after the object is placed in proximity to the device.

15. The method of claim 14, further comprising the steps of:
heating the object; and
determining that the object is a non-biological object by noting an inverse change in the light intensity after the object is placed in proximity to the device, compared to the change in light intensity by the biological object.

16. The method of claim 14, wherein the step of determining the object is a biological object comprises:
determining that the object is a biological object by measuring a change in the light intensity within about 100 seconds after the object is placed in proximity to the device.

17. The method of claim 13, further comprising the steps of:
measuring no substantial change in the light intensity after the object is placed in proximity to the device, thereby determining that the object is a non-biological object;
withdrawing the non-biological object from proximity of the device;
placing the non-biological object in proximity to a biological object for a period of time; and
measuring a change in the light intensity after the non-biological object is placed in proximity to the device.

18. The method of claim 13, further comprising the step of:
controlling the effect on the measured light intensity from the sensor by at least one of the following parameters: temperature, electromagnetic radiation dependence, chemical interactions, and mechanical interactions.

19. A method of measuring the activity of a biological object comprising the steps of:
measuring a background light intensity of a biological measurement system comprising:
a light source;
a sensor proximate to and disposed at an angle from the light source;
a covering material disposed above the sensor;
a photodetector proximate to the sensor and disposed at an angle relative to the light source;
a non-light transmitting partition disposed between the light source and the photodetector, the partition configured to isolate the photodetector from the light source; and
a non-light transmitting housing encasing the light source, sensor, and photodetector;
placing an object in non-contact proximity to the device; and
determining if the object has biological properties by viewing a video monitor.

20. A method of measuring the activity of a biological object comprising the steps of:
measuring a background light intensity of a biological measurement system comprising:
a light source;
a sensor proximate to and disposed at an angle from the light source;
a covering material disposed above the sensor;
a video camera proximate to the sensor and disposed at an angle relative to the light source;
a non-light transmitting partition disposed between the light source and the video camera, the partition configured to isolate the video camera from the light source; and
a non-light transmitting housing encasing the light source, sensor, and video camera;
placing an object in non-contact proximity to the device; and
determining if the object has biological properties by viewing a video monitor.
